(19) United States
(12) Patent Application Publication
(10) Pub. No.: US 2020/0046918 A1
(43) Pub. Date: Feb. 13, 2020

(54) SPRAY DELIVERY DEVICE

(71) Applicant: The Technology Partnership plc, Melbourn Hertfordshire (GB)

(72) Inventor: Robert Gordon Maurice Selby, Melbourn Hertfordshire (GB)

(21) Appl. No.: 15/735,078

(22) PCT Filed: Jun. 10, 2016
(86) PCT No.: PCT/GB2016/051712

§ 371 Date: Dec. 8, 2017

(30) Foreign Application Priority Data
Jun. 11, 2015 (GB) 1510166.0

(51) Int. Cl. A61M 15/00; B05B 17/06

(52) U.S. Cl. CPC A61M 15/0066; A61M 2016/0033; A61M 15/0085; B05B 17/0669

(57) ABSTRACT

There is disclosed a device for delivering a fluid spray to a mouth of a user, either directly, or via a flow conductor such as a tube or pipe. The spray device uses a vibrating perforate membrane to create the spray and the vibration of the membrane is controlled in response to input from a flow rate sensor which detects a flow rate through the device or through the flow conductor. There is further disclosed a corresponding method of controlling a spray head in a spray delivery device.

### FIELD OF THE INVENTION

This invention relates to electronic spray devices in which a perforate membrane is vibrated to generate liquid droplets. In particular, the invention relates to methods for enabling a delivery rate of the device to be tailored to the required rate, by reference to a demand signal from a suitable sensor.

### BACKGROUND

For inhalation devices such as electronic cigarettes the need for sensing flow to trigger smoke or droplet generation is well known. For example US2012186594 describes using a sensor to sense both the flow direction and rate of inhaled air. This sensor signal is used to determine when the air flow is in the right direction and exceeds a set threshold rate so as to trigger smoke generation.

There is a benefit in matching the delivery rate of any fluid, or medicament, to be delivered to a user, to suit the inhalation rate to provide a better user experience and also to maximise the efficiency of medicament/inhalant delivery. Similarly, for coating processes where droplet delivery is used to coat substrates, matching the delivery rate to a sensed property of the receiving substrate can optimise the quality of the coating and minimise wastage of the coating material. Furthermore when treating gas streams other than inhaled air matching the delivery rate to suit the gas flow rate will minimise cost and maximise benefits of the treating process if a sensor is used to determine the required delivery rate.

Electronic droplet generators that use ultrasonic vibration to generate liquid droplets are well known and have found use in a wide range of fields including medical drug delivery and the treatment of air or gas (for example fragrance delivery and humidification) and for coating of surfaces such as application of treatments to skin or industrial processes such as painting or applying surface treatments to components.

A preferred embodiment of such ultrasonic spray devices is one in which the perforate membrane itself is vibrated by the driver element (commonly called the actuator or vibrator element) with examples including U.S. Pat. No. 4,533,082 and EP 0431992. This enables the delivery of relatively well monodispersed droplets without requiring the pressure waves to be transmitted through a liquid layer. A preferred embodiment of such a device is described in U.S. Pat. No. 5,518,179 uses a bending mode actuator to deliver the vibrational energy to the membrane as this enables the use of thin low cost actuators.

Electronic spray technologies by definition require a power source and electronic circuitry (henceforth referred to as a spray controller) to be incorporated or linked to the spray generator.

Spray generators such as those described above often have a resonant frequency at which energy is efficiently transferred to the perforate membrane and hence to the liquid. To obtain good performance it is known that the spray generator must be operated at or at least close to the resonant frequency (EP 1,731,228 for example).

For such devices, the vibration is often generated by applying an alternating voltage across a unimorph or bimorph piezoceramic component or similar. The alternating voltage drives this component into oscillatory deformation at the drive frequency. This deformation is coupled to the perforate membrane causing it to vibrate and generate the liquid spray. Thus the characteristics of the input electrical waveform have a direct bearing on the spray that is generated.

### SUMMARY OF THE INVENTION

The invention provides a device for delivering a fluid spray to a mouth of a user, either directly, or via a flow conductor such as a tube or pipe. The spray device uses a vibrating perforate membrane to create the spray and the vibration of the membrane is controlled in response to input from a flow rate sensor which detects a flow rate through the device or through the flow conductor. The invention can be realised using the following features, any or all of which can be implemented in any combination to achieve the desired effects. The invention further provides a corresponding method of controlling a spray head in a spray delivery device.

According to the invention there is provided a spray delivery device for delivering a fluid spray to a fluid flow conductor, comprising:
a spray generator,
a spray controller; and
an air flow sensor;
wherein the spray generator comprises a perforate membrane and actuation means configured to ultrasonically vibrate the perforate membrane in response to a drive signal from the spray controller, such that vibration of the perforate membrane causes liquid droplets to be ejected from an ejection side of the perforate membrane; and
wherein the flow sensor is configured to provide a flow signal representative of an air flow rate through the flow conductor, such that the spray controller can modulate a spray rate of the spray generator in response to the sensed air flow rate.

The flow signal may be proportional to the air flow rate or may be proportional to a function of the air flow rate.

The spray controller may be configured to generate no spray when the sensed air flow is below a predetermined threshold value.

The spray controller may be configured to modulate the drive signal using time based modulation.

The spray controller may be configured to modulate the drive signal by amplitude based modulation.

The spray controller may be configured to modulate the drive signal by shifting a frequency of the drive signal away from the resonant frequency of the device, such that for the same drive signal amplitude, a lower power is delivered by the spray generator.

The spray controller may be configured to modulate the drive signal by adjusting the mark space ratio of the drive signal.

The spray controller may be configured to adjust the mark space ratio of the drive signal such that the spray head drive is switched on and off at a frequency different from the resonant drive frequency.

The spray controller may be configured to adjust the mark space ratio of the drive signal such that the spray head drive is switched on and off at a frequency lower than the resonant drive frequency.

The spray controller may be configured to adjust the mark space ratio of the drive signal such that the spray head drive is switched on and off at a sufficient rate to provide at least 5 switching cycles within a 2 second time period.

The spray controller may be configured to multiply the flow signal by a proportionality constant, k, to derive the drive signal amplitude.

The device may be configured to permit a user to change the proportionality constant, k.

The controller may be configured to adjust the proportionality constant k over time to reduce the rate of dose delivery for a given inhalation rate over time.

The device may be configured to adapt the proportionality constant, k in response to the flow signal.

The device may be configured to adapt the proportionality constant, k, over a plurality of uses of the device, to adapt the delivery profile to suit the user's inhalation.

The device may be configured to adapt the proportionality constant, k, such that a defined dose is delivered substantially evenly within the user's typical inhalation time.

The device may be configured to adapt the proportionality constant, k, such that a defined dose is delivered substantially evenly within a predefined sub-portion of the user's total inhalation time.

The invention further provides an inhalation device comprising a spray delivery device according to any of the preceding claims, wherein the spray delivery device is configured to deliver the fluid spray to a fluid flow path through a body of the inhalation device.

The inhalation device may be configured for delivering nicotine based formulations or may be an electronic cigarette.

The invention further provides a device for delivering a fluid spray to a mouth of a user, either directly, or via a flow conductor such as a tube or pipe, the device comprising a vibratable perforate membrane for generating the spray, wherein the device is configured to control the vibration of the membrane in response to input from a flow rate sensor which detects a flow rate through the device or through the flow conductor.

The invention further provides a method of controlling a spray head in a spray delivery device, the spray head comprising a vibratable perforate membrane for generating a spray, the method comprising receiving a sensed air flow signal representative of an air flow rate through a flow conductor to which the spray head is arranged to deliver a spray, and modulating a spray rate of the spray generator in response to the sensed air flow signal.

### BRIEF DESCRIPTION OF THE FIGURES

Embodiments of the invention will now be described with reference to the following figures in which:

FIG. 1A shows a schematic representation of a device according to an embodiment of the present invention;

FIG. 1B shows a schematic representation of a device according to a further embodiment of the present invention;

FIG. 2 illustrates a first sensor signal and corresponding membrane drive signal for implementation in an embodiment of the invention;

FIG. 3 illustrates a second sensor signal and corresponding membrane drive signal for implementation in an embodiment of the invention;

FIG. 4 illustrates a mark space moderator signal and sensor signal for implementation in an embodiment of the invention;

FIG. 5 illustrates a further mark space moderator signal and sensor signal for implementation in an embodiment of the invention;

FIG. 6 illustrates a sensor signal and corresponding drive signal for implementation in an embodiment of the invention; and

FIG. 7 illustrates a further example of a sensor signal and a corresponding drive signal for implementation in an embodiment of the invention.

### DETAILED DESCRIPTION OF EMBODIMENTS OF THE INVENTION

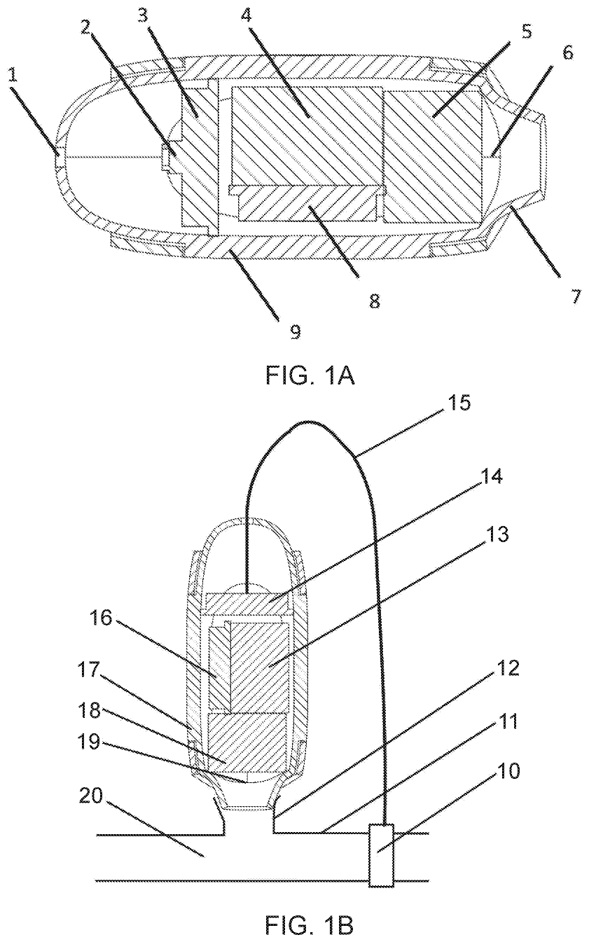

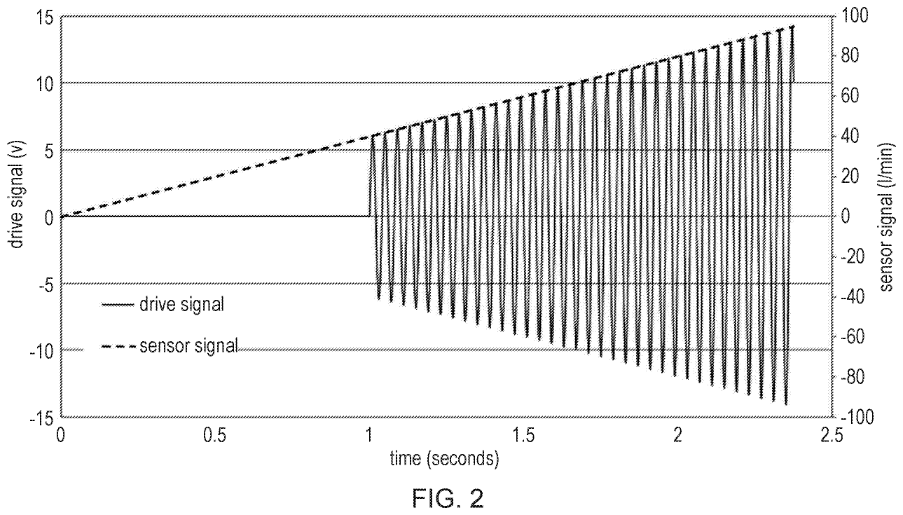

FIG. 1A shows a schematic representation of an inhalation device according to an embodiment of the invention. The device has means to sense the inhaled airflow rate, preferably provided through the combination of the flow restrictor provide by opening (1) serving as a pressure restriction. The opening is typically of a cross sectional area of 6 to 12 mm² and the pressure sensor (2), preferably mounted on a PCB (3). The combination of the mouthpiece (7) and body (9) may form an otherwise sealed chamber with the exception of the opening (1) and the opening of the mouthpiece (7). When the user inhales the pressure drop induced by drawing air through (1) is measured by the pressure sensor. The signal from the pressure sensor (2) is connected to and measured by the controller (microcontroller) (4). In turn the flow rate of air is calculated by the controller (4) and the appropriate delivery rate of fluid into the flow passing through the inhaler is determined for the measured inhalation rate. The controller then generates a drive signal to drive the atomising head (6). Variation of the drive signal allows adaption of the delivery rate to the inhalation rate by the means described in relation to FIGS. 2 to 7. The controller (4) and PCB (3) are supplied with energy from the battery (8). The drive signal generated by the controller (4) is supplied to the perforate membrane based resonant atomising head (6) which in turn is mounted in a fluid reservoir (5). The resonant atomising head itself will typically consist of a PZT ceramic actuator bonded to a metallic substrate such that when the actuator is excited by an appropriate frequency alternating signal will vibrate and induce vibration in the perforate membrane.

An alternative configuration can be envisaged in which the spray delivery device is arranged adjacent a flow conductor such as a pipe, tube or other channel for conducting a fluid flow, preferably a gas flow such as air. The spray delivery device can be arranged to deliver spray into the fluid flow. A flow sensor can be configured to measure a flow in the flow conductor, and the spray device can be configured to deliver a fluid into the flow as a spray in response to the output of the flow sensor as described in the following. It will therefore be apparent that the delivery device and control means described herein can be used in an inhalation device or in a spray delivery device configured to deliver a spray into a flow conductor with which the spray device is placed in fluid communication.

An example of such an arrangement is shown in FIG. 1B. In this figure the flow conductor (20) has a flow sensor (10) such as that produced by Honywell S&C part number AWM720P1. This flow sensor provides a signal that is fed into the spray device (17) where the device, preferably on a daughter board (14), processes the flow signal to control the delivery of the spray. The drive PCB (13) and battery (16) in turn supply the necessary drive signal to the spray head (19), which delivers droplets at the appropriate rate from the fluid reservoir (18). The droplets are fed into the air stream in the flow conductor (20) via the port (12). Any of the drive methods discussed in the following can of course be used in either of the devices of FIGS. 1A and 1B.

Tuning the drive signal to the actuator can be achieved by a spray controller scanning a pre-programmed frequency band before commencing spraying, and using the results of this scan to record the resonant frequency of the spray generator and to use it as a later reference frequency. In this case the resonant frequency can be periodically checked by the controller whilst spraying so as to capture any shifts in resonant frequency due to changes in liquid loading for example. Alternatively a self-resonant drive circuit can be used in which the spray generator forms part of a resonant circuit such that the drive signal frequency is automatically tuned to the resonant frequency of the head.

The combination of an efficient and effective spray generator and drive electronics can allow high delivery rates to be achieved. This then offers the potential for tuning the fluid delivery rate to suit the specific and changing needs of the application. Typical applications that can benefit from adjusting the fluid flow or delivery rate in response to a variable parameter include: matching the fluid delivery rate to a flow rate of a gas stream, in the case of dosing the gas stream with an additional fluid, for example adding water droplets to allow control of humidity of a gas stream once the droplets evaporate; and adding anaesthetic agents to a gas stream in proportion to a flow rate of the gas. Further applications include drug delivery applications such as a nebuliser, inhaler or similar drug delivery device or consumer drug delivery device including nasal sprays and similar over the counter (OTC) devices where the delivery rate of a drug is moderated in proportion to the to the inhalation rate of the user.

For drug delivery applications, the flow rate could be proportional to the inhalation rate, optionally subject to a minimum trigger level below which delivery is not started.

Methods of Adjusting the Delivery Rate

The output from the drive electronics for an ultrasonic spray generator can be adjusted in a number of ways to modulate the delivery rate. These methods include changing the output voltage, changing the drive frequency to be slightly offset from resonance, and using pulse-width modulation, as some examples.

The amplitude of vibration of a resonant, but damped, device can be controlled by the level or amplitude of the drive signal. Therefore, reducing the output voltage has the effect of reducing the number of nozzles on the spray head which are vibrating with sufficient acceleration to generate droplets, and so this reduces the flow rate. This method can provide progressive control of the flow rate within some limits. For example, below a certain drive voltage the flow rate will reduce very dramatically, as very many fewer nozzles will vibrate sufficiently to emit droplets. However this method has the disadvantage of needing a means to control the output drive voltage, which will typically require additional electronic components.

Similarly the amplitude of vibration is affected by how close frequency of stimulation is to the resonant frequency of the device. Adjusting the drive frequency to be slightly off the resonant frequency, for example by being offset from the resonant frequency by amounts such as 500 Hz to a few KHz away from the true resonant frequency. By characterising the behaviour of the actuator the degree of flow rate attenuation for a given offset from resonance allows the control of flow rate to be achieved and can therefore provide a second way in which to control the vibration and hence flow rate from such spray generators. This method has the advantage of using largely the same electronic components as those needed for basic driving of the spray generator, rather than needing additional components to adjust the drive voltage.

In addition to modification of frequency and/or voltage, another way to impact spray performance is through the use of time-based modulation of the drive signal, which we shall call “duty cycling”. For pressurised sprays in industrial environments, pulsing of the spray by turning a valve on and off rapidly is used to adjust flow rate by such time-based modulation. This is commonly referred to as pulse width modulation. In general, flow rate is linearly proportional to on-time, thus a reduction in duty cycle from 100% (constantly on) to 50% (on half the time) would approximately halve the flow rate. The controller of FIG. 1 can therefore be configured to implement such a time-based modulation of the drive signal for the device of FIG. 1.

It is non-obvious that this approach would work with an electronic spray, as there is no valve to switch and the drive signal oscillates at high frequency to drive the spray generation process. However, it has been demonstrated that time-based modulation of this drive signal can be used to adjust the average flow rate of an electronic spray device. This approach works by applying the high frequency drive signal in bursts with gaps of no, or reduced, signal in between. The acceptable frequency of switching and the limits of on and off periods depend on application.

For example, for consumer applications, and in electronic cigarettes in particular, perception is critical. For some uses the overall period of this drive regime (burst time plus gap time) must be short enough that the plume appears to be continuous; whereas in some applications a slightly staccato delivery, having distinguishable gaps between bursts, may be appealing. In the case of micro-processor controlled drive electronics, the control parameters are set in firmware and so can be changed to suit user preference. In this case the frequency of pulse switching could be changed or, for example, for an electronic cigarette type device, the scaling between inhalation rate and delivery rate could be adjusted to adapt the delivery rate to the inhalation preference for the user. In one example we can consider the fluid delivery rate as being proportional to the inhaled flow rate, i.e. to the sensed gas flow rate:

Delivery rate=k×inhalation rate

where k is the proportionality factor. If k is increased, the dose rate, or fluid delivery rate, for a given inhalation rate will increase and vice versa. A similar technique could be used to gradually reduce the dose over time, through an inbuilt algorithm provided in the spray controller, by reducing the value of k so that the user's dose could be gradually reduced, either during a single inhalation, or over multiple inhalations over an extended period of time.

The appearance of continuous delivery generally requires the overall period of the duty cycle, or otherwise stated, the time between each instance of the modulation signal switching from off to on, to be less than approximately 30 milliseconds, more ideally, less than 15 milliseconds. For dosing applications, such as treating a gas stream in a pipe, for example, for humidification or anasethetic applications, the acceptable limits for switching can have longer on and off periods, as mixing within the gas stream will average out the concentration and, further, the delivery will not be directly visible to the user. Therefore, the appearance of the delivery is less important. Given that a typical breath may be 2 seconds long, to provide the effect of proportional control, the rate at which changes can be made to the delivery rate is important. With a mark space ratio switching period of 15 milliseconds, over 100 periods of switching are available, giving a sufficiently high temporal resolution of on-off switching to give the impression of continuous variability. In this way, the speed of response of the ultrasonic spray device allows almost real time matching of dosing requirements to the demand signal from the sensor. The fluid delivery rate can also be adapted such that the total on-time within the user's typical inhalation time is sufficient to deliver a specific dose. The drive sequence for modulating the drive signal to create the desired fluid delivery rate can be iteratively adapted over a sequence of inhalation profiles. If the controller records the duration and total air volume of one or more successive inhalation events, the controller can establish a nominal inhalation duration and air volume for one or more of the recorded inhalation events. This can be based on the latest previous inhalation, or on a moving average of a number of previous inhalation events. Using this nominal inhalation duration, the controller can determine a proportionality constant K, such that the rate of delivery is tuned to be completed within the expected inhalation event. For example if the user typically inhales 2 liters of air in a 2 second inhalation and the set dose volume is 15 microliters then an appropriate K value can be calculated. For simplicity if the inhalation rate is assumed to be constant for the two seconds and the delivery rate is similarly assumed to be constant for the delivery duration the delivery rate would be 7.5 microliters per second and:

$$k = \frac{aveage inhalation rate}{delivery rate}$$

So the appropriate K value would be 0.125 microliters per seconds per liter per minute of inhaled air.

By setting the K values in this way the controller can tune the actual delivery rate to suit the inhalation rate which will in practice vary through the inhalation. Increasing K by a few percent will ensure that the dose is delivered before the end of the inhalation. By successively adjusting the K value in this way over inhalation events allows tuning of the delivery to the anticipated inhalation profile for the user. This can all act to refine the delivery profile, such that the dose is delivered in a desired manner during a typical inhalation of a user. The chosen profile may be set to deliver the fluid evenly over the user's typical inhalation time. The profile may alternatively be adapted such that the fluid delivery is targeted for a particular sub-section of the user's typical inhalation, for example to ensure delivery to the deep lung by increasing the K value to ensure that the dose is delivered before the end of the inhalation event

In matching the required delivery rate to a measurable property, the performance of the overall system can be optimised. In the case of a drug delivery device, the dosing rate can be controlled to stay within tolerable limits or to optimise the delivery rate to suit the inhalation rate. This can be useful in the case of delivering a drug to patients with a low inspiration flow such as COPD [Chronic obstructive pulmonary disease] patients or children where the maximum delivery rate can be overwhelming. Similarly in self titration applications it has been found that users have a preference for the delivery rate to be in proportion to the inhalation rate. Expressed simply, the harder the user inhales the greater the delivery rate. The use of a pressure restriction and a pressure sensor can be used to create a measure of breath inhalation rate. For example, in an inhaler type device with a restriction providing a flow resistance of

$$0.2 \frac{\sqrt{Mbar}}{l min} ,$$

combined with a pressure sensor such as the amplified pressure sensor offered by Sensor Technics part number HDIM200DBE8H5, the inhalation rate can be measured and used to provide a signal to control the delivery rate.

The total dose volume can be controlled by the user controlling the inhalation duration and rate. Additionally or alternatively, a metering function can limit the maximum dose to be delivered before the device is re-primed by a user, or permitted to be re-primed, by the controller. In treating gas streams or setting the dose rate for a drug delivery device, an appropriate sensor such as a flow sensor may provide sufficient accuracy of demand signal. It may be further necessary to measure temperature and/or humidity to more fully characterise the target delivery rate.

FIG. 2 illustrates an example of the modulation of a drive signal by the spray controller of the invention in response to a measured flow signal coming from the flow sensor of the device. The figure shows moderation of a sinusoidal drive signal, such as may be used for driving an ultrasonic actuator of the device of FIG. 1, in response to a demand signal shown as a linearly rising signal. Typical drive frequencies for the ultrasonic spray generator may be in the range 30 kHz to 200 kHz and more typically in the range 50 to 120 kHz. For clarity the drive signal is not shown with its real frequency in this figure.

FIG. 2 also illustrates a deferred reaction to the demand signal, such that the drive signal is only initiated by the spray controller once the demand signal reaches a particular trigger threshold. In the case of inhalation through a drug delivery device typical value for the trigger threshold is 10 to 15 liters per minute. This serves as a good compromise between wanting the delivery to start early in the inhalation and providing a sufficiently robust sensor value to trigger, avoiding false triggering due to sensor signal noise or air movements due to factors other than inhalation occurring. Given that the time for inhalation rates to reach 10 or 15 liters per minute at the start of an inhalation is typically 0.1 seconds or sometimes shorter, this trigger level makes good use of the available inhaled volume. This has the advantage of avoiding false triggering events and also, in the case of amplitude modulation, prevents trying to drive the spray head with too low a voltage for effective operation.

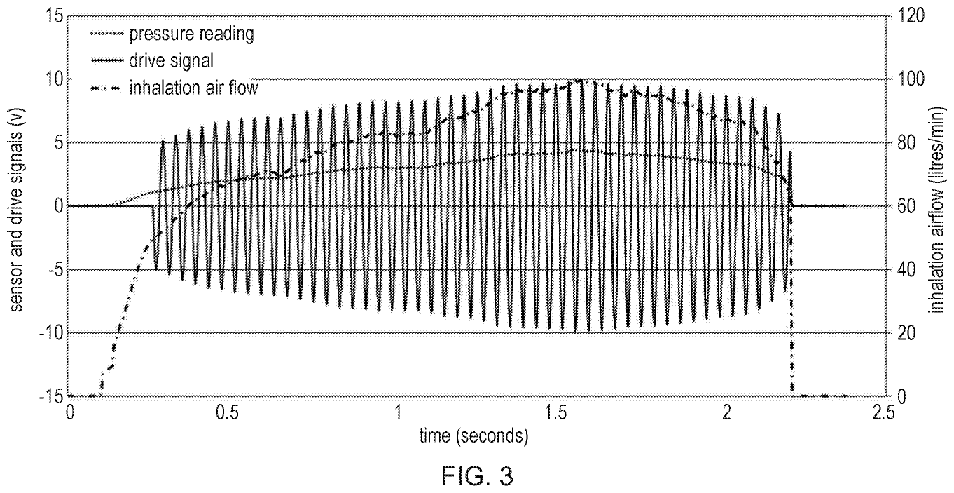

FIG. 3 shows a similar arrangement, preferably where the demand signal is representative of an inhalation through a relatively high resistance drug delivery device, such as an inhaler or electronic cigarette. This implementation will tend to give a longer duration of an inhalation, such as the 2 seconds or thereabouts shown in the Figure. As in FIG. 2, the drive signal is moderated in amplitude in proportion to the demand signal, which is the output of the flow sensor, and there is a defined minimum threshold trigger level, below which the drive signal is not initiated, which threshold may be at any of the values described above.

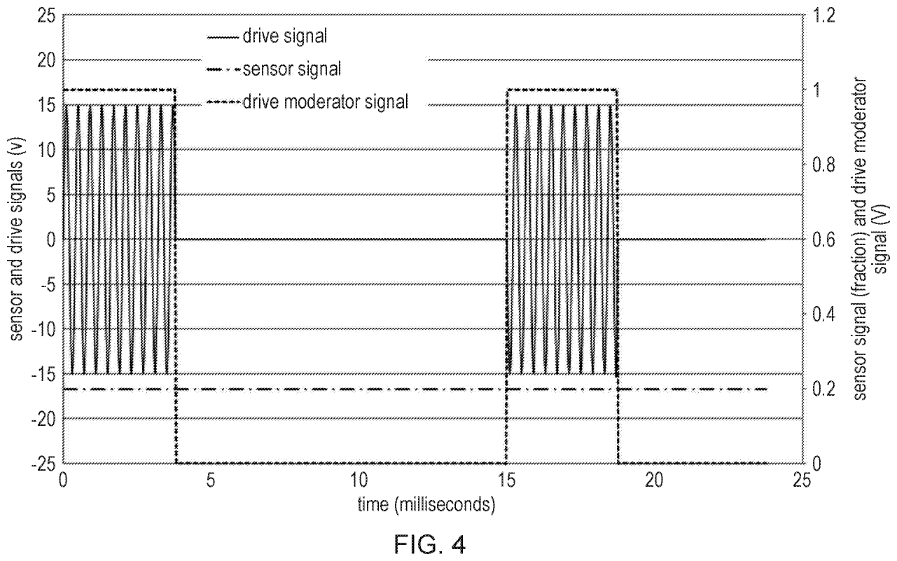

FIG. 4 is an illustration of pulse width modulation of a drive waveform in response to a demand signal. The typical drive signal frequency may be 100 kHz and the period for the pulse width modulation may be in the range 5 to 30 ms. As such, the scales for these Figures have been adjusted to provide legibility and to illustrate the technique. The graph of FIG. 4 shows the sensor or demand signal and the drive signal being on for approximately 4 ms and off for approximately 11 ms. This effects a 36% duty cycle in terms of both the time over which the spray generator is driven and in terms of the resulting time averaged fluid flow delivery rate.

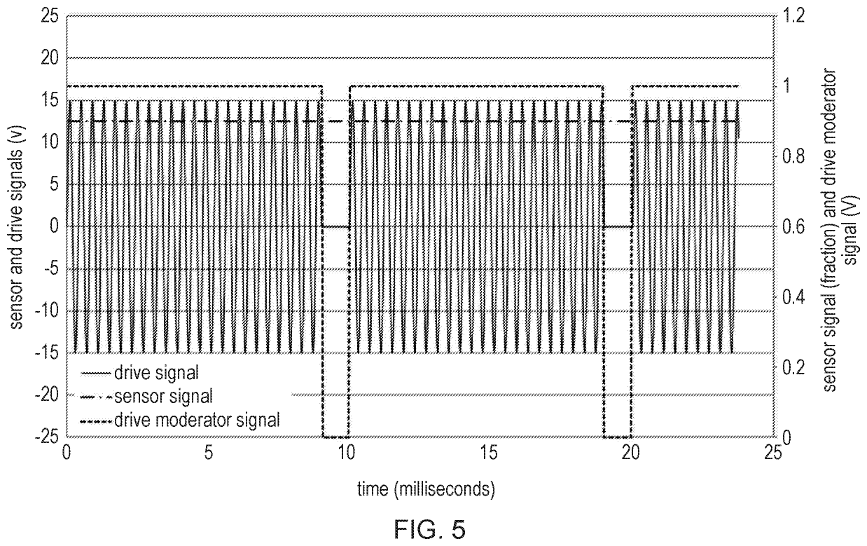

FIG. 5 shows a similar arrangement, where the demand is higher and so the mark space ratio for the drive signal is higher. The graph shows the sensor or demand signal and the drive signal being on for approximately 8 ms and off for approximately 2 ms, an 80% duty cycle. Varying the duty cycle in this way has been shown to alter the delivery rate linearly with respect to the mark space ratio.

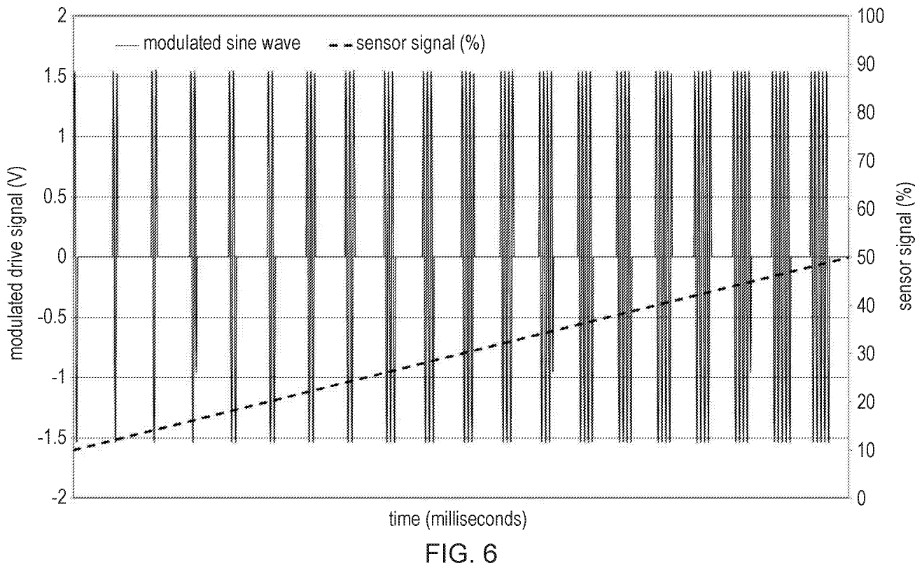

FIG. 6 shows mark space modulation of the drive signal in response to a demand signal that is increasing linearly. The mark space ratio increases in a linear fashion in response to the rising sensor signal value and so matches the delivery rate to the demand.

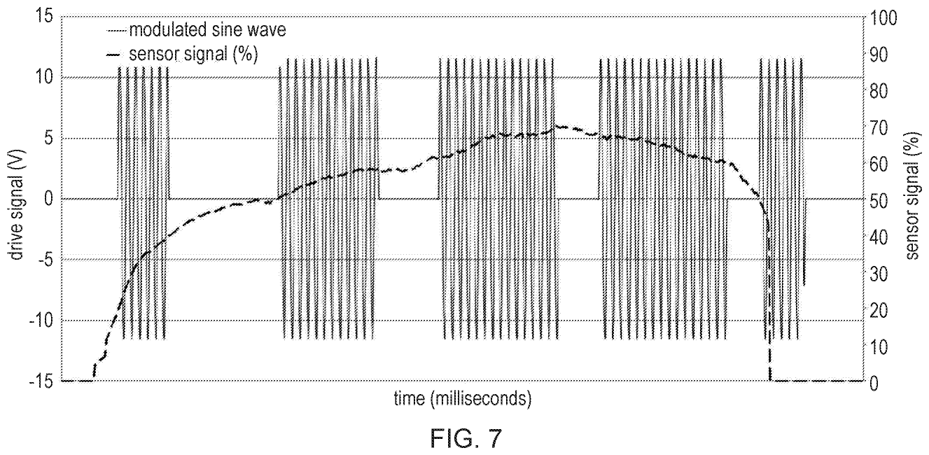

FIG. 7 shows mark space modulation in response to inhalation breath profile signal, again periods of higher demand signal result in a higher mark space ratio and hence higher delivery rate.

In setting a mark space ratio for the FIGS. 4 and 5 the inhaled air flow values may be in the range 0 to 100 liters per minute and if the controller is set to drive the head 100% of the time when the sensed air flow is 100 liters per second and proportionally across the range 0 to 100 liters per minutes e.g. for 50 liters per minute the duty cycle would be 50% and hence the mark space ratio would be set to 1:1 so on for 1 time unit and off for 1 time unit. The period of switching that set the duration of the on and off periods can be set to choice as discussed above, so typically for an inhalation device the switching period may be set to 15 ms and hence a 1:1 mark space ratio would result in driving the head for 7.5 ms and not driving the head for 7.5 ms. Similarly for a 75 liter per minute air flow the mark space ratio would be 3:4 and the on time would be 11.25 ms and the off time would be 3.75 ms within a switching period. In FIG. 4 the flow signal is 0.25 (25%) of full scale value set for the controller. The switching period has been set to 15 ms, so that the on time is 20% of 15 ms, so 3 ms, and the off time is 12 ms. In the example shown in FIG. 5 the switching period has set at 10 ms and the sensed flow rate is 90% of the maximum expected values. Hence the on time is 9 ms and the off time is 1 ms.

FIG. 6 shows the effect of the controller responding to a linearly increasing sensor signal (e.g. air flow). Again, the controller has been set to expect air flows up to 100 liters/minute and sets the mark space ratio in the proportion that the air flow rate is to the maximum values. By taking an average flow value within the switching period the controller calculated the mark space ratio appropriate for that switching period. So in the first switching period the flow average is 11 liters per minute (and 11% of maximum) so the mark space ratio is set to 11%. Consequently the on time is 1.1 ms and the off time is 8.9 ms. At the right hand end of the graph the average flow rate is 50 liters per second (or 50% of the maximum expected value) and the on time is set to 5 ms and the off time is similarly 5 ms.

In FIG. 7 the configuration is similar to that of FIG. 6. The average measured flow rate in a switching period is determined and this is used to set the mark space ratio. In the first switching cycle at the left hand side of the graph the average flow rate is 30% of the maximum and so the on time is 30% of the switching period (3 ms) and the off time is 70% of the switching period (7 ms). In the 4^{th} switching period the average measured flow is 80% of maximum and so the on time 8 ms (80% of the switching period) and the off time is 2 ms.

1. A spray delivery device for delivering a fluid spray to a fluid flow conductor, comprising:
a spray generator,
a spray controller; and
an air flow sensor;
wherein the spray generator comprises a perforate membrane and actuation means configured to ultrasonically vibrate the perforate membrane in response to a drive signal from the spray controller, such that vibration of the perforate membrane causes liquid droplets to be ejected from an ejection side of the perforate membrane; and
wherein the flow sensor is configured to provide a flow signal representative of an air flow rate through the flow conductor, such that the spray controller can modulate a spray rate of the spray generator in response to the sensed air flow rate.

2. A device according to claim 1, wherein the flow signal is proportional to the air flow rate.

3. A device according to claim 1, wherein the flow signal is proportional to a function of the air flow rate.

4. A device according to claim 1, wherein the spray controller is configured to generate no spray when the sensed air flow is below a predetermined threshold value.

5. A device according to claim 1, wherein the spray controller is configured to modulate the drive signal using time based modulation.

6. A device according to claim 1, wherein the spray controller is configured to modulate the drive signal by amplitude based modulation.

7. A device according to claim 1, wherein the spray controller is configured to modulate the drive signal by shifting a frequency of the drive signal away from the resonant frequency of the device, such that for the same drive signal amplitude, a lower power is delivered by the spray generator.

8. A device according to claim 1, wherein the spray controller is configured to modulate the drive signal by adjusting the mark space ratio of the drive signal.

9. A device according to claim 8, wherein the spray controller is configured to adjust the mark space ratio of the drive signal such that the spray head drive is switched on and off at a frequency different from the resonant drive frequency.

10. A device according to claim 8, wherein the spray controller is configured to adjust the mark space ratio of the drive signal such that the spray head drive is switched on and off at a frequency lower than the resonant drive frequency.

11. A device according to claim 8, wherein the spray controller is configured to adjust the mark space ratio of the drive signal such that the spray head drive is switched on and off at a sufficient rate to provide at least 5 switching cycles within a 2 second time period.

12. A device according to claim 1, wherein the spray controller is configured to multiply the flow signal by a proportionality constant, k, to derive the drive signal amplitude.

13. A device according to claim 12, wherein the device is configured to permit a user to change the proportionality constant, k.

14. A device according to claim 12, wherein the spray controller is configured to adjust the proportionality constant k over time to reduce the rate of dose delivery for a given inhalation rate over time.

15. A device according to claim 12, wherein the device is configured to adapt the proportionality constant, k in response to the flow signal.

16. A device according to claim 15, wherein the device is configured to adapt the proportionality constant, k, over a plurality of uses of the device, to adapt the delivery profile to suit the user's inhalation.

17. A device according to claim 16, wherein the device is configured to adapt the proportionality constant, k, such that a defined dose is delivered substantially evenly within the user's typical inhalation time.

18. A device according to claim 17, wherein the device is configured to adapt the proportionality constant, k, such that a defined dose is delivered substantially evenly within a predefined sub-portion of the user's total inhalation time.

19. An inhalation device comprising a spray delivery device according to claim 1, wherein the spray delivery device is configured to deliver the fluid spray to a fluid flow path through a body of the inhalation device.

20. An inhalation device according to claim 19, wherein the inhalation device is configured for delivering nicotine based formulations.

21. A device for delivering a fluid spray to a mouth of a user, either directly, or via a flow conductor such as a tube or pipe, the device comprising:
a vibratable perforate membrane for generating the spray, wherein the device is configured to control the vibration of the membrane in response to input from a flow rate sensor which detects a flow rate through the device or through the flow conductor.

22. A method of controlling a spray head in a spray delivery device, the spray head comprising a vibratable perforate membrane for generating a spray, the method comprising:
receiving a sensed air flow signal representative of an air flow rate through a flow conductor to which the spray head is arranged to deliver a spray, and
modulating a spray rate of the spray generator in response to the sensed air flow signal.
